(19) United States
(12) Patent Application Publication
(10) Pub. No.: US 2021/0345707 A1
(43) Pub. Date: Nov. 11, 2021

(54) DISINFECTING FACE MASK

(71) Applicants: Hosam Tleibi, Brampton (CA); Ghena Al Zayed, Brampton (CA)

(72) Inventors: Hosam Tleibi, Brampton (CA); Ghena Al Zayed, Brampton (CA)

(21) Appl. No.: 16/867,425

(22) Filed: May 5, 2020

(51) Int. Cl. A41D 13/11; A62B 23/02; A62B 18/02; A61L 9/20

(52) U.S. Cl. CPC A41D 13/1192; A62B 23/02; A62B 7/10; A61L 9/20; A62B 18/025

(57) ABSTRACT

A device for filtering and disinfecting; the device includes a face mask, a disinfecting unit, and a power source to power the device. The disinfecting unit includes a plurality of light emitting diodes (LEDs) emitting short wave ultraviolet (UV) light. The device is useful for filtering air prior to being breathed in by a wearer of the face mask and disinfecting the face mask and/or a filter thereof via the short wave UV light.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of breathing devices of existing art and more specifically relates to an air filtering and disinfecting face mask.

### RELATED ART

Ultraviolet germicidal irradiation (UVGI) is a method of disinfection utilizing short-wavelength ultraviolet light to kill or inactivate microorganisms such as bacteria, viruses, molds and other pathogens. This is achieved by destroying nucleic acids and disrupting their DNA, leaving them unable to perform vital cellular functions.

Face masks such as respirators, surgical masks, cloth masks, etc. are known in the art and are often used to protect a wearer from bacteria, viruses, dust, pollution and the like. Most of these face masks include filtering means to filter air before it is breathed in by the wearer. The filtering means either have to be replaced after time, or the mask has to be washed or disposed of This can get costly. Thus, there exists a need for an effective means of disinfecting face masks. A suitable solution is desired.

### SUMMARY OF THE INVENTION

In view of the foregoing disadvantages inherent in the known breathing device art, the present disclosure provides a novel disinfecting face mask. The general purpose of the present disclosure, which will be described subsequently in greater detail, is to provide an air filtering face mask having a disinfecting unit built therein to disinfect the face mask and/or a filter attached thereto.

A device for filtering and disinfecting is disclosed herein. The device includes a face mask which may include a mask-body configured to cover at least a mouth and nose of a wearer. The mask-body may include at least one filter means being configured to filter air passing through the mask-body before it enters the mouth and nose of the wearer. Further, a fastener may be attached to the mask-body and configured to removably fasten the face mask to a head of the wearer.

A disinfecting unit may be provided on the device and may include a unit-body attached to the mask-body and at least one ultraviolet light source attached to the unit-body and in communication with the at least one filter means. The at least one ultra-violet light source may be configured to emit short-wave ultraviolet light that is configured to disinfect the at least one filter means. At least one power source may be provided and configured to supply power to the device.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a disinfecting face mask, constructed and operative according to the teachings of the present disclosure.

FIG. 1 is a front side view of the device for filtering and disinfecting during an ‘in-use’ condition, according to an embodiment of the disclosure.

FIG. 2A is a front view of the device for filtering and disinfecting according to an embodiment of the present disclosure.

FIG. 2B is a side view of the device for filtering and disinfecting according to an embodiment of the present disclosure.

FIG. 3A is a front view of the device for filtering and disinfecting according to an embodiment of the present disclosure.

FIG. 3B is a side view of the device for filtering and disinfecting according to an embodiment of the present disclosure.

FIG. 4 is a front view of the device for filtering and disinfecting according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to face masks or breathing devices and more particularly to a disinfecting face mask as used to filter air prior to being breathed in by a wearer of the face mask, and disinfect the face mask and/or a filter thereof.

Generally disclosed is a device including a face mask and a disinfecting means, the face mask being configured to filter air and the disinfecting means being configured to provide ongoing disinfection to the face mask. The device may include a plurality of ultraviolet (UV) light emitting diodes (LEDs) attached to a UV LED holder attached to the face mask. A UV sensor may further be provided on the UV LED holder and in communication with the UV LEDs. The UV sensor may be configured to measure UV light strength.

Further, a logic circuit may be provided that is configured to scan through the UV LEDs and turn on one or more LEDs at a time, thus reducing power consumption and making the device more efficient. The logic circuit may be set at a fixed scan or may include a BLUETOOTH® connection that may be controlled via a software application downloadable to a smartphone, tablet computer, etc. The logic circuit may communicate UV light strength measured by the UV sensor to the software application.

A power source may be provided to provide power to the UV LEDs and the logic circuit. The power source may include disposable batteries or an external power bank connectable to the device via a micro universal serial bus (USB) connector port located on the device. Further, an on/off switch may be provided on the device to selectively turn the power source on or off.

There may be two different versions of the device: a reusable mask version, and a disposable mask version. The reusable mask version may include the logic circuit being disposed within the face mask. The disposable mask version may include the logic circuit external to the device and connectable to the device via the micro USB connector port.

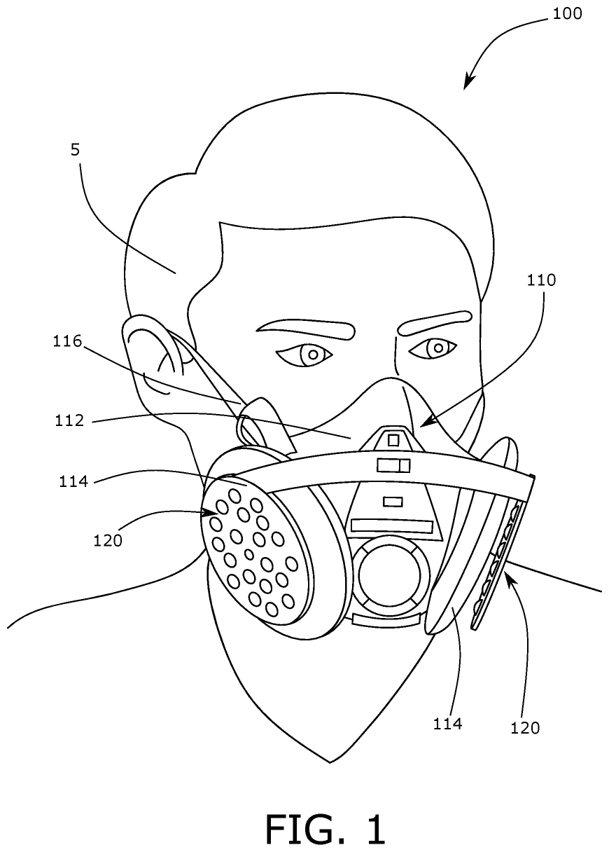

Referring now more specifically to the drawings by numerals of reference, there is shown in FIGS. 1-4, various views of a device 100.

FIG. 1 shows a device 100 during an ‘in-use’ condition, according to an embodiment of the present disclosure. Here, the device 100 may be beneficial for filtering and disinfecting. As illustrated, the device 100 may include a face mask 110, a disinfecting unit 120 and at least one power source 130 configured to supply power to the device 100. A wearer 5 is shown wearing the device 100 according to an embodiment of the present disclosure. As shown, the face mask 110 may include a mask-body 112 configured to cover at least a mouth and nose of the wearer 5 and a fastener 116 attached to the mask-body 112 configured to removably fasten the face mask 110 to a head of the wearer 5. Further, the face mask 110 may include at least one filter means 114 attached to the mask-body 112 configured to filter air passing through the mask-body 112 before it enters the mouth and nose of the wearer 5.

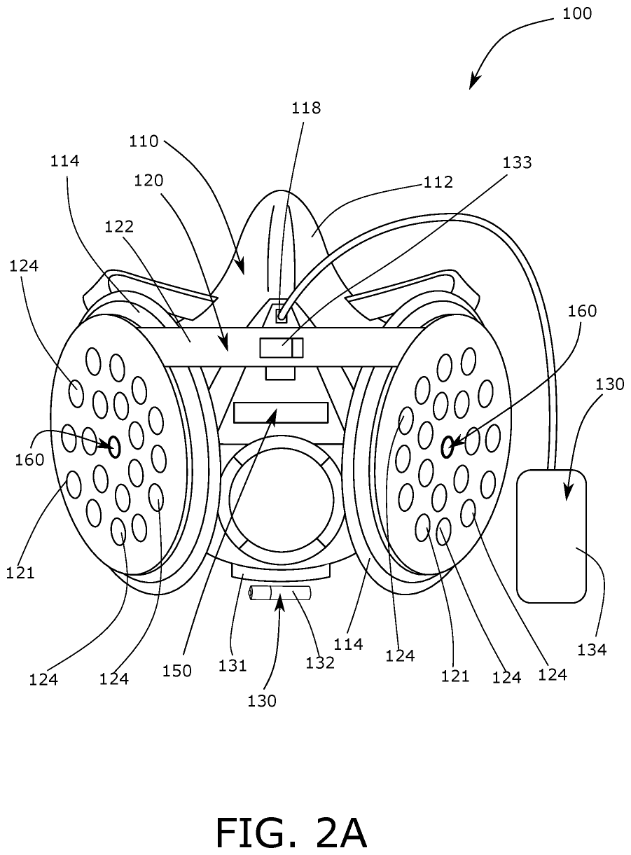

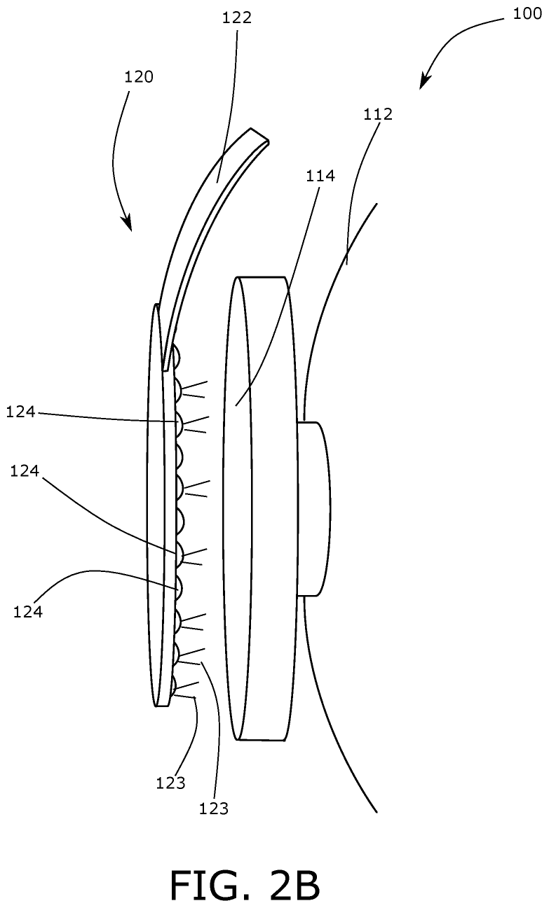

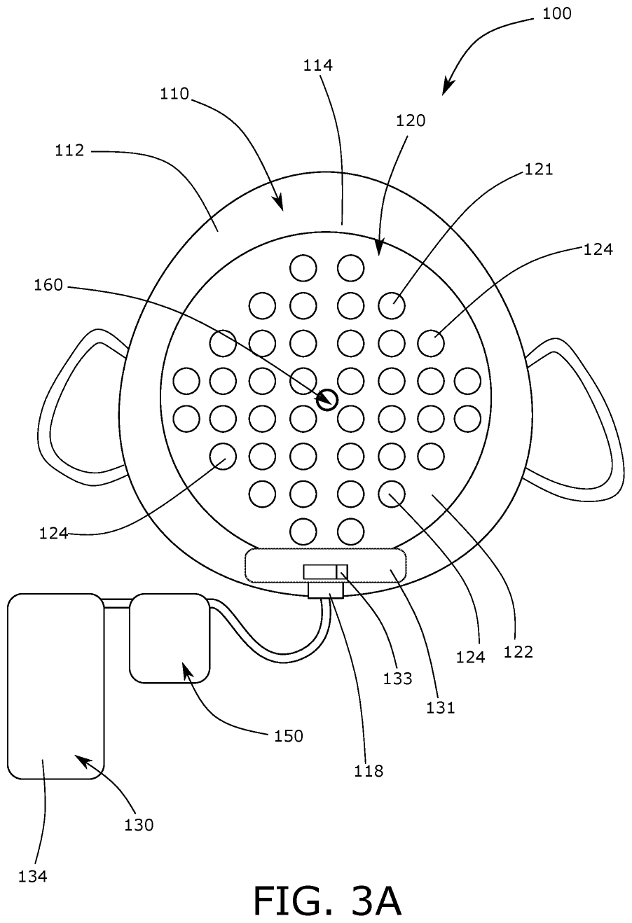

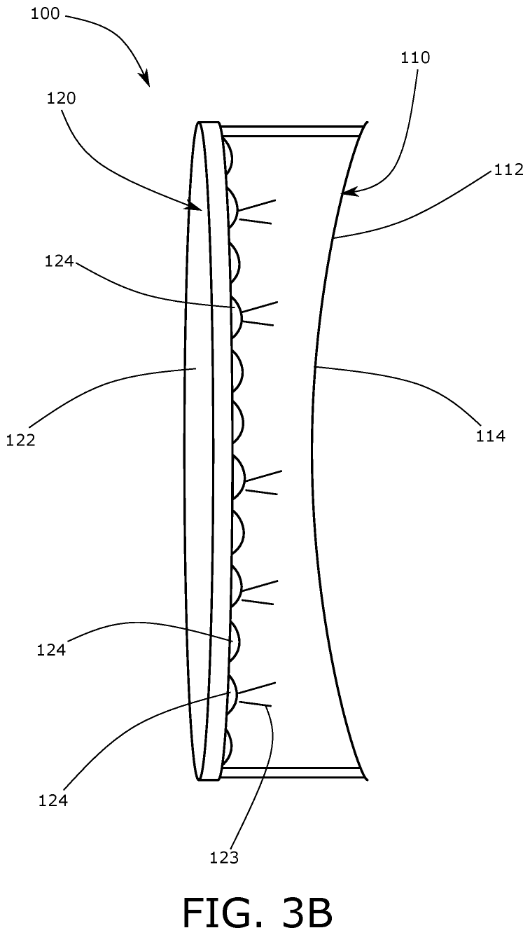

Referring now to FIGS. 2A-3B showing various views of the device 100 of FIG. 1, according to an embodiment of the present disclosure. As shown in FIGS. 2A-2B, the at least one filter means 114 may be two filters. Shown in FIG. 2A is an air purifying respirator. In some embodiments, the at least one filter means 114 may be attached about the mask-body 112 as with air purifying respirators like is shown in FIGS. 2A-2B. In other embodiments, the at least one filter means 114 may be attached to the mask-body 112 as an additional layer as with respirators such as N95 masks, filtering facepiece particle masks, etc. In other embodiments, the at least one filter means 114 may be integral to the mask-body 112. For example, the at least one filter means 114 may be an inherent feature of the mask-body 112, such as surgical masks, cloth masks, homemade masks, disposable masks, etc. as is shown in FIG. 3A.

The disinfecting unit 120 may include a unit-body 122 attached to the mask-body 112. The unit-body 122 may be removable from the mask-body 112, or may be permanently attached. and at least one ultraviolet light source 121 attached to the unit-body 122 and in communication with the at least one filter means 114. The at least one ultra-violet light source may be configured to emit short-wave ultraviolet light 123 and the short-wave ultraviolet light 123 may be configured to disinfect the at least one filter means 114 via ultraviolet germicidal irradiation (UVGI) (FIG. 2B and FIG. 3B). The short-wave ultraviolet light 123 may be ultraviolet C (UVC). Preferably, the at least one ultraviolet light source 121 may be a plurality of light emitting diodes 124. The light emitting diodes 124 may emit the short-wave ultraviolet light 123 at wavelengths between 255-280 nanometers (nm). Whilst light emitting diodes are a preferred embodiment, other ultraviolet light sources may be contemplated such as mercury-based lamps or pulsed-xenon lamps.

A logic circuit 150 may be provided and in communication with the plurality of light emitting diodes 124. In one embodiment, as shown in FIG. 2A, the logic circuit 150 may be disposed within the face mask 110. This may be a reusable version of the device 100. In addition, the device 100 may further comprise at least one sensor 160 in communication with the logic circuit 150. The at least one sensor 160 may be attached to the unit-body 122 and may be configured to measure an intensity of the short-wave ultraviolet light 123 emitting from each of the plurality of light emitting diodes 124.

The logic circuit 150 may be configured to receive intensity information from the at least one sensor 160 and in response to the intensity information, the logic circuit 150 may be configured to selectively power at least one of the light emitting diodes 124 on and off. For example, if the at least one sensor 160 senses that the intensity of the short-wave ultraviolet light 123 is too strong (or other signal), the at least one sensor 160, via the intensity information it sends, may tell the logic circuit 150 to turn off one or some of the light emitting diodes 124. Likewise, if the at least one sensor 160 senses that the intensity of the short-wave ultraviolet light 123 is too weak, the at least one sensor 160, via the intensity information it sends, may tell the logic circuit 150 to turn on one or some of the light emitting diodes 124. This may be useful in maintaining an intensity sufficient to disinfect the at least one filter means 114, whilst preserving maximum power.

The at least one power source 130 may provide power to the logic circuit 150 and the light emitting diodes 124. Further, as shown, the device 100 may include a power switch 133 for selectively turning the at least one power source 130 on and off. In some embodiments, the at least one power source 130 may include at least one battery 132 attached about the face mask 110. The device 100 may include a battery holder 131 attached to the face mask 110 and configured to house the at least one battery 132. The at least one battery 132 is shown to be outside of the battery holder 131 in FIG. 2A for illustrative purposes. The at least one battery 132 may be at least one disposable battery or at least one rechargeable battery. The rechargeable battery may be removable and replaceable, or the rechargeable battery may be charged via a charging port and charging cable. As shown, the device 100 may further include a micro universal serial bus connector port 118 disposed within the mask-body 112. The rechargeable battery may be charged via the micro universal serial bus connector port 118.

In some embodiments, the at least one power source 130 may include a power bank 134 configured for connection to the device 100 via the micro universal serial bus connector port 118. The power bank 134 may be included as well as the at least one battery 132, or one may be provided over another. Further, in some embodiments, as shown in FIG. 3A, the logic circuit 150 may be external to the device 100 and configured for connection to the device 100 via the micro universal serial bus connector port 118. This may be a disposable or semi-disposable version of the device 100. Those with ordinary skill in the art will now appreciate that upon reading this specification and by their understanding the art of powering, sensing, disinfecting as described herein, methods of powering, sensing, disinfecting will be understood by those knowledgeable in such art.

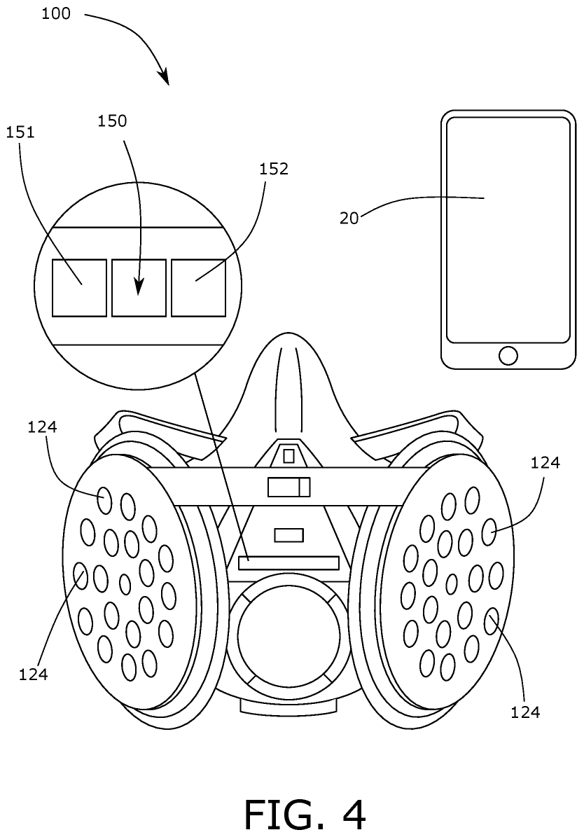

Referring now to FIG. 4 showing a front view of the device 100. In some embodiments, the device 100 may include a wireless transceiver 152 configured to communicate with an external device 20 such as a smartphone, computer, tablet computer, etc. In some embodiments, the wireless transceiver 152 may be BLUETOOTH® enabled. Further, the device 100 may include a processor 151 configure to facilitate communication between the logic circuit 150 and the external device 20. In some embodiments, the logic circuit 150 may include the processor 151. In other embodiments, the processor 151 may be separate. In some embodiments, the device 100 may further include a software application downloadable to the external device 20. The wireless transceiver 152 may connect the device 100 with the external device 20 and the processor 151 may allow a user to control the plurality of light emitting diodes 124 via the software application.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for filtering and disinfecting comprising:
a face mask, the face mask including:
a mask-body, the mask-body being configured to cover at least a mouth and nose of a wearer, the mask-body including at least one filter means, the at least one filter means being configured to filter air passing through the mask-body before it enters the mouth and nose of the wearer; and
a fastener attached to the mask-body, the fastener being configured to removably fasten the face mask to a head of the wearer;
a disinfecting unit, the disinfecting unit including:
a unit-body attached to the mask-body; and
at least one ultraviolet light source attached to the unit-body and in communication with the at least one filter means, the at least one ultra-violet light source configured to emit short-wave ultraviolet light, the short-wave ultraviolet light being configured to disinfect the at least one filter means; and
at least one power source configured to supply power to the device.

2. The device of claim 1, wherein the at least one ultraviolet light source comprises a plurality of light emitting diodes.

3. The device of claim 2, further comprising a logic circuit in communication with the plurality of light emitting diodes.

4. The device of claim 3, wherein the logic circuit is disposed within the face mask.

5. The device of claim 3, further comprising at least one sensor configured to measure an intensity of the short-wave ultraviolet light emitting from each of the plurality of light emitting diodes, and wherein the at least one sensor is in communication with the logic circuit.

6. The device of claim 5, wherein the at least one sensor is attached to the unit-body.

7. The device of claim 6, wherein the logic circuit is configured to receive intensity information from the at least one sensor and in response to the intensity information the logic circuit is configured to selectively power at least one of the light emitting diodes on and off.

8. The device of claim 1, further comprising a wireless transceiver configured to communicate with an external device.

9. The device of claim 8, further comprising a processor configured to facilitate communication between the logic circuit and the external device.

10. The device of claim 1, wherein the at least one power source includes at least one battery attached about the face mask.

11. The device of claim 10, further comprising a battery holder attached to the face mask, and wherein the power source holder is configured to house the at least one battery.

12. The device of claim 10, further comprising a micro universal serial bus connector port disposed within the face mask.

13. The device of claim 12, wherein the at least one power source includes a power bank configured for connection to the device via the micro universal serial bus connector.

14. The device of claim 12, wherein the logic circuit is external to the device and configured for connection to the device via the micro universal serial bus.

15. The device of claim 1, wherein the at least one filter means comprises two filters.

16. The device of claim 1, further comprising a power switch for selectively turning the power source on and off.

17. A device for filtering and disinfecting comprising:
a face mask, the face mask including:
a mask-body, the mask-body being configured to cover at least a mouth and nose of a wearer;
two filters attached to the mask-body, each of the two filters being configured to filter air passing through the mask-body before it enters the mouth and nose of the wearer; and
a fastener attached to the mask-body, the fastener being configured to fasten the face mask to a head of the wearer;
a disinfecting unit, the disinfecting unit including:
a unit-body attached to the mask-body; and
a plurality of light emitting diodes attached to the unit-body and in communication with the two filters, the plurality of light emitting diodes configured to emit short-wave ultraviolet light, the short-wave ultraviolet light being configured to disinfect the two filters;
a micro universal serial bus connector port disposed within the face mask;
at least one sensor attached to the unit-body, the sensor configured to measure an intensity of the short-wave ultraviolet light emitting from each of the plurality of light emitting diodes;
a logic circuit disposed within the face mask, the logic circuit in communication with the at least one sensor and the plurality of light emitting diodes, the logic circuit configured to receive information from the sensor and in response to the intensity information the logic circuit is configured to selectively power at least one of the light emitting diodes on and off;
at least one power source configured to supply power to the device; and
a power switch for selectively turning the power source on and off.

18. The device of claim 17, further comprising a wireless transceiver and a processor, the wireless transceiver being configured to communicate with an external device, the processor being configured to facilitate communication between the logic circuit and the external device.

19. A device for filtering and disinfecting comprising:
a face mask, the face mask including:
a mask-body, the mask-body being configured to cover at least a mouth and nose of a wearer;
at least one attached to the mask-body, the at least one filter means being configured to filter air passing through the mask-body before it enters the mouth and nose of the wearer; and
a fastener attached to the mask-body, the fastener being configured to fasten the face mask to a head of the wearer;
a disinfecting unit, the disinfecting unit including:
a unit-body attached to the mask-body; and
a plurality of light emitting diodes attached to the unit-body and in communication with the at least one filter means, the plurality of light emitting diodes configured to emit short-wave ultraviolet light, the short-wave ultraviolet light being configured to disinfect the at least one filter means;
a micro universal serial bus connector port disposed within the face mask;
at least one sensor attached to the unit-body, the sensor configured to measure an intensity of the short-wave ultraviolet light emitting from each of the plurality of light emitting diodes;
a logic circuit external to the device and configured for connection to the device via the micro universal serial bus, the logic circuit in communication with the at least one sensor and the plurality of light emitting diodes, the logic circuit configured to receive intensity information from the sensor and in response to the intensity information the logic circuit is configured to selectively power at least one of the light emitting diodes on and off;
at least one power source configured to supply power to the device; and
a power switch for selectively turning the power source on and off.

20. The device of claim 19, further comprising a wireless transceiver and a processor, the wireless transceiver being configured to communicate with an external device, the processor being configured to facilitate communication between the logic circuit and the external device.
